(19) United States
(12) Patent Application Publication
(10) Pub. No.: US 2017/0252429 A1
(43) Pub. Date: Sep. 7, 2017

(54) Direct Method for providing Immunological Memory against HIV

(71) Applicant: Kareem Thomas Robinson, Baltimore, MD (US)

(72) Inventor: Kareem Thomas Robinson, Baltimore, MD (US)

(73) Assignee: Kareem Thomas Robinson, Baltimore, MD (US)

(21) Appl. No.: 15/426,055

(22) Filed: Feb. 7, 2017

Related U.S. Application Data
(60) Provisional application No. 62/304,234, filed on Mar. 6, 2016.
(60) Provisional application No. 62/326,859, filed on Apr. 25, 2016.

(51) Int. Cl. A61K 39/21; C12N 5/0783; C07K 16/10; C12N 5/0781

(52) U.S. Cl. CPC A61K 39/21; C12N 5/0635; C12N 5/0637; C07K 16/1045; C12N 2501/998; A61K 2039/5158

(57) ABSTRACT

The disclosed method provides a direct method for creating immunological memory against HIV by educating a patient's naive T and B lymphocytes in vitro into HIV antigen-specific adaptive immune memory lymphocytes. Said memory lymphocytes which were created in vitro undergo process wherein memory B lymphocytes are genetically modified for broadly neutralizing antibodies, as well as repeatedly stimulated with HIV antigens. Memory T lymphocytes undergo gene editing for chemokine receptors used by HIV for infection. Said memory lymphocytes are administered to said patient with an immunological memory against HIV in a solution which does not contain chemicals that are opposed to anti-vaccine supporters.

### CROSS-REFERENCE TO RELATED APPLICATIONS

This application claims the benefit of U.S. provisional patent application Ser. No. 62/304,234, filed 2016 Mar. 6 by the present inventor.

This application claims the benefit of U.S. provisional patent application Ser. No. 62/326,859, filed 2016 Apr. 25 by the present inventor.

### RELATED U.S. PATENT DOCUMENTS

| Application Number | Filing Date | Pat. No. | Issue Date |
|---|---|---|---|
| 62/304,234 | Mar. 06, 2016 | | |
| 62/326,859 | Apr. 25, 2016 | | |

### REFERENCES CITED [REFERENCED BY]

### U.S. Patent Documents

| | | |
|---|---|---|
| 7,030,228 | April 2006 | Schmitz et al. |
| 7,067,134 | June 2006 | Kang et al. |
| 7,608,273 | October 2009 | Kang et al. |
| 8,323,961 | December 2012 | Nabel et al. |
| 8,785,411 | July 2014 | Narayan et al. |
| 8,840,890 | September 2014 | Lewis et al |
| 9,273,118 | March 2016 | Beaumont et al. |
| 9,376,471 | June 2016 | Weiner et al. |
| 9,415,099 | August 2016 | Pavlakis et al. |

### OTHER REFERENCES

Balla-Jhagjhoorsingh, Sunita S. et al. “Characterization of Neutralizing Profiles in HIV-1 Infected Patients from Whom the HJ16, HGN194 and HK20 mAbs Were Obtained.” Ed. Lisa Ng Fong Poh. PLoS ONE 6.10 (2011): e25488. PMC. Web. 10 Sep. 2016.
Cell expansion (n.d.). GE Healthcare Life Sciences. Retrieved Jan. 4, 2017 from https://promo.gelifesciences.com/GL/XURI/expansion.html#.WG2KMqOZPUo
Janeway C A Jr, Travers P, Walport M, et al. Immunobiology: The Immune System in Health and Disease. 5th edition. New York: Garland Science; 2001. Immunological memory. Available from: https://www.ncbi.nlm.nih.gov/books/NBK27158/
Janeway C A Jr, Travers P, Walport M, et al. Immunobiology: The Immune System in Health and Disease. 5th edition. New York: Garland Science; 2001. B-cell activation by armed helper T cells. Available from: https://www.ncbi.nlm.nih.gov/books/NBK27142/
Klein, F., Halper-Stromberg, A., Horwitz, J. A., Gruell, H., Scheid, J. F., Bournazos, S., . . . Nussenzweig, M. C. (2012). HIV therapy by a combination of broadly neutralizing antibodies in humanized mice. Nature, 492(7427), 10.1038/nature11604. http://doi.org/10.1038/nature11604
Liao, H.-X., Lynch, R., Zhou, T., Gao, F., Alam, S. M., Boyd, S. D., . . . Haynes, B. F. (2013). Co-evolution of a broadly neutralizing HIV-1 antibody and founder virus. Nature, 496(7446), 469-476. http://doi.org/10.1038/nature12053
Mahmood, T., & Yang, P.-C. (2012). Western Blot: Technique, Theory, and Trouble Shooting. North American Journal of Medical Sciences, 4(9), 429-434. http://doi.org/10.4103/1947-2714.100998
Origene Technologies Inc. Retrieved January 4, 2017, from: http://origene.com/CRISPR-CAS9/
Origene Technologies Inc. Retrieved Jan. 9, 2017 from: origene.com/search/ProductList.aspxkeyword=variable+region&go.x=0&go.y=0.
Origene Technologies Inc. Retrieved Jan. 9, 2017 from: www.origene.com/orf/?utm_source=Bing&utm_medium=cpc&utm_term=cDNA%2Bclone&utm_content=cDNA%2B clones&utm_campaign=Top%2BKeywords
Patient Centered Medicine 2: Basic Screening Physical Examination. lumen, n.d. Web, 4 Jan. 2017, http://www.meddean.luc.edu/lumen/meded/ipm/Ipm2/BSE_laminated%20card.pdf
Rosette Sep: Unique Immunodensity Cell Isolation. (n.d.). Stem Cell Technologies Inc. Retrieved Jan. 4, 2016 from: https://www.stemcell.com/products/brands/rosettesep.html#
World Health Organization, WHO guidelines on drawing blood: best practices in phlebotomy, 2010. Web, 4 Jan. 2017 from: http://apps.who.int/iris/bitstream/10665/44294/1/9789241599221_eng.pdf

### TECHNICAL FIELD

The present invention relates to a new preventative HIV Vaccine.

### BACKGROUND

Since HIV discovery, scientists have scrambled to develop an effective vaccine to prevent HIV progression to Aids. It was immediately discovered that the virus high mutation rate was a challenge to a successful vaccine. After years of research, the discovery of broadly neutralizing antibodies gave hope that a vaccine is possible. These antibodies neutralize various strains/clades of HIV, and memory lymphocytes that would develop from vaccination need the ability to create these powerful antibodies upon an initial HIV infection. Despite numerous methods, scientists have failed to create an effective vaccine that can establish the immune memory cells in a patient which can produce these critical antibodies upon a natural infection.

Several methods of HIV vaccination have been proposed—for example, U.S. Pat. No. 8,323,961 to Nabel, et al. (2012) which employs the use of viral vectors carrying the genetic information for HIV. Clinical trials such as RV144 have demonstrated that although the use of viral vectors has elicited an immune response, it did not elicit neutralizing antibodies upon natural infection. DNA vaccines utilize nucleic acid as an immunogen see, for example, U.S. Pat. No. 9,415,099, U.S. Pat. No. 8,785,411, and U.S. Pat. No. 9,376,471. Clinical trials such as HVTN 505 showed that even though DNA vaccines do elicit an immune response, its secondary response against an initial infection with said pathogen is relatively weak. Current methods of vaccination that have worked successfully against most pathogens are inefficient against HIV, and nevertheless, all HIV vaccine models heretofore known suffer from disadvantages:
(a) Humoral immune secondary response established by current models have shown to only produce non-neutralizing antibodies upon a natural HIV infection. Most vaccines that have effectively prevented infection produce neutralizing antibodies against their targeted pathogen, and an effective HIV vaccine requires an immediate neutralizing antibody response against said pathogen upon an initial infection.
(b) HIV vaccine models often fail in establishing both humoral and cell-mediated immunological memory against said pathogen. Usually, a weak humoral immunological memory is established without cell-mediated immunological memory.
(c) Current models propose an indirect approach in establishing immunological memory. Indirect because immunogens that were administered to the patient are assumed to have established an effective immunological memory that can elicit neutralizing antibodies upon a natural HIV infection with no way of knowing directly.
(d) Some models proposed include genetically modified killed HIV pathogens as an immunogen. Fear of infection from certain public factions will render this vaccine model ineffective for the establishment of herd immunity against said virus.
(e) Models proposed includes the use of chemicals such as thiomersal, aluminum, and formaldehyde in the vaccine solution. Certain factions of the public fear said chemicals associated with the solution of vaccines and refuse vaccination for themselves or family members.

### SUMMARY

In one embodiment, the invention serves three purposes. (a) It provides HIV antigen-specific memory lymphocytes needed for immunological memory. (b) It provides immune memory lymphocytes needed to elicit a secondary neutralizing antibody immune response against HIV. (c) It provides a means of vaccinating concerned citizens who refuse vaccination due to fear of certain chemicals in its solution or of getting infected by said pathogen.

In embodiments monocytes, naive T lymphocytes, and naive B lymphocytes are isolated from patient's PBMCs and cultured in vitro.

In embodiments, memory B lymphocytes are stimulated with HIV antigens repeatedly until the desired antibody affinity for its antigenic target is received.

In certain embodiments, B cells are genetically modified with genes for HIV antibodies.

In certain embodiments, memory CD4 T lymphocytes genes to CCR5 and/or gene to CXCR4 surface protein chemokine receptor is knocked/suppressed before administration to subject.

In embodiments, HIV memory lymphocytes are created from patient's isolated lymphocytes and are administered to a patient.

Accordingly, in aspects, the invention provides a subject HIV antigen-specific memory B lymphocytes with the genetic potential for broadly neutralizing HIV antibodies, neutralizing HIV antibodies, and/or non-neutralizing HIV antibodies upon a natural infection with HIV.

In another aspect, the invention provides HIV antigen-specific memory CD4 helper T cells.

In yet another aspect, the invention provides HIV antigen-specific memory CD8 cytotoxic T cells.

### BRIEF DESCRIPTION OF THE DRAWINGS

FIGS. 1 & 2 shows an overall description of the processes involved in memory lymphocyte production.

FIGS. 3 & 4 shows briefly the concept of training and genetically modifying memory lymphocytes before administration.

### DETAILED DESCRIPTION

The practice of the present invention employs unless otherwise indicated conventional techniques of molecular biology, genetics, microbiology, cell biology, and immunology which are within the skill of the art. Techniques such as cellular isolation, cellular expansion, and genetic modification/editing are provided through commercial kits unless otherwise indicated. Usage of said techniques is explained in the literature/directions provided from vendors.

### Definition

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention. To facilitate the understanding of this invention, a number of terms are defined below. Terms defined herein have meanings as commonly understood by a person of ordinary skill in the areas relevant to the present invention.

Terms such as “a”, “an” and “the” are not intended to refer to only a singular entity but include the general class of which a specific example may be used for illustration. The terminology herein is used to describe specific embodiments of the invention, but their usage does not delimit the invention, except as outlined in the claims.

According to the present invention, the general use herein of the term “antigen” refers; to any portion of an HIV protein (peptide), partial protein, and full-length protein wherein the protein is naturally occurring, synthetically made or a glycoprotein wherein the glycoprotein contains a carbohydrate portion of the antigen and epitopes of glycoproteins. An antigen can be as small as a single epitope, a single immunogenic domain or larger, and can include multiple epitopes or immunogenic domain. Antigens can be modified at any portion of an HIV protein, partial protein, epitopes, domains, and full-length protein wherein the protein is synthetically made. An antigen may be nucleic acids which encode genes for HIV proteins.

According to the present invention, antibodies or immunoglobulins are referred to antibodies from mammalian species primarily humans where the antibodies (IgM, IgG, IgD, IgA, IgE) are naturally occurring from B cells. An antibody may be non-neutralizing, neutralizing, or broadly neutralizing. Non-neutralizing antibodies function to tag the virus for destruction. Neutralizing antibodies limits the effectiveness of the virus preventing it from infecting healthy cells and are very specific to a particular epitope. Broadly neutralizing antibodies target various strains of HIV due to the unchanging region of the targeted epitopes.

According to the present invention, a subject is a mammalian species primarily a human.

As used herein, the term “cytokine” refers to any one of the numerous factors that exert a variety of effects on cells, for example, inducing growth or proliferation. Non-limiting examples of cytokines which may be used alone or in combination in the practice of the present invention include, interleukin-2 (IL-2), interleukin-4 (IL-4), interleukin-6 (I1-6), interleukin-7 (IL-7), interleukin-10 (IL-10), interleukin-15 (IL-15), G-C SF, granulocyte macrophage-colony stimulating factor (GM-CSF), macrophage-colony stimulating factor (M-CSF), b-cell activating factor (BAFF) and CD40 ligand. Cytokines are commercially available from several vendors. It is intended, although not always explicitly stated, that molecules having similar biological activity as wild-type or purified cytokines (e.g., recombinantly produced or muteins thereof) are intended to be used within the spirit and scope of the invention.

The terms “major histocompatibility complex” or “MHC” refers to a complex of genes encoding cell-surface molecules that are required for antigen presentation to T cells and rapid graft rejection. The proteins encoded by the MEW are known as “MEW molecules” and are classified into Class I and Class II MHC molecules.

The term “genetically modified” means containing and/or expressing a foreign gene or nucleic acid sequence which in turn, modifies the genotype or phenotype of the cell or its progeny. In other words, it refers to any addition, deletion or disruption of a cell's endogenous nucleotides.

The term HIV refers to a lentivirus known as Human Immunodeficiency Virus. HIV consist of two species, HIV 1 and HIV 2 with a variety of subtypes and different clades in those subtypes. It refers to either live wild-type strain or killed versions of the pathogen.

The term “antigen presenting cells (APCs)” refers to cells capable of presenting one or more antigens in the form of peptide-MHC complex recognizable by immune effector cells of the immune system, and thereby inducing an adaptive immune response against the antigen or antigens being presented. APCs such as macrophages, B-cells, activated T-cells, and dendritic cells; or other molecules, naturally occurring or synthetic, such as purified MHC Class I & II are capable of presenting antigens in the form of proteins, protein epitopes, nucleic acids. However, only dendritic cells have the capacity to present antigens in an efficient amount to activate naive T-cells for cytotoxic T-lymphocyte (CTL) responses.

The term monocyte refers to a diverse population of similar white blood cells found circulating in peripheral blood. Monocytes have the ability to differentiate into macrophages and dendritic cells.

The term patient refers to the subject receiving treatment which is primarily a human.

The term “macrophage” refers to a diverse population of morphologically similar cells found in a variety of tissues. It is a major APC for the organism. Macrophages can be generated from monocytes in vitro, and are an optional APC for the invention.

The term “dendritic cells (DCs)” refers to a population of morphologically similar cell types of myeloid and lymphoid origins. Dendritic cells constitute the most potent and preferred APCs in the organism. While the dendritic cells can be differentiated from monocytes, they possess distinct phenotypes.

The term “adaptive immune lymphocytes” refers to a diverse population of B cells from the humoral immune system, and T cells from the cellular-mediated immune system. B cells are the humoral immune cells that create antibodies after they have differentiated into plasma cells. T cells from the cell-mediated immune system consist of diverse populations primarily of CD4 and CD8 T cells. CD4s or helper T cells help orchestrate the immune response, while CD8 or cytotoxic T cells kill infected cells with active MHC 1 on their plasma membrane.

The term T lymphocytes (T cells) are distinguished from other lymphocytes by the T cell receptor (TCR), which B cells and Natural Killer cells do not express. There are several types of T cells based on their specific function: helper/effector, cytotoxic, memory, regulatory and gamma delta (γδ) T cells. When a pathogen is detected, information is communicated to T cells through antigen presentation from antigen presenting cells. Following activation, naive CD4+ T cells differentiate into one of the several lineages of T helper cells (Th1, Th2, Th9, Th17, or Th22), depending primarily on the antigen, the strength of the TCR signal, and the cytokines present in the surrounding extracellular environment. Differentiation of each T cell subset is associated with the secretion of a defined array of cytokines that orchestrate a directed response to the antigen.

The term B lymphocytes (B cells) refers to a diverse population of the humoral immune cells. B cells are responsible for generating antibodies to specific antigens, which they bind via B cell receptors (BCR). Activation of B cells occurs via antigen recognition by BCRs and a required co-stimulatory, secondary activation signal provided by either helper T cells or the antigen itself. This results in stimulation of B cell proliferation and the differentiation of B cells into plasma cells or memory B cells. Following the primary immune response, a small number of B lymphocytes develop into memory B lymphocytes, which express high-affinity surface immunoglobulins (mainly IgG), survive for a longer period of time, and enable a rapid secondary response.

The term “antigen-specific memory lymphocyte” refer to adaptive immune cells that are capable of a secondary immune response to a previously encountered antigen. The antigen-specific memory cells will be obtained in vitro from the invention and will have a memory for the various epitopes contained on the numerous protein antigens of HIV. The memory cells can be resident, effector, follicular, or central memory lymphocytes.

The term pathogen refers to the disease-causing organism known as HIV.

As used herein, the term in vitro refers to a process taking place in culture dishes or test tubes outside of a living organism in a laboratory or controlled environment.

### Overview

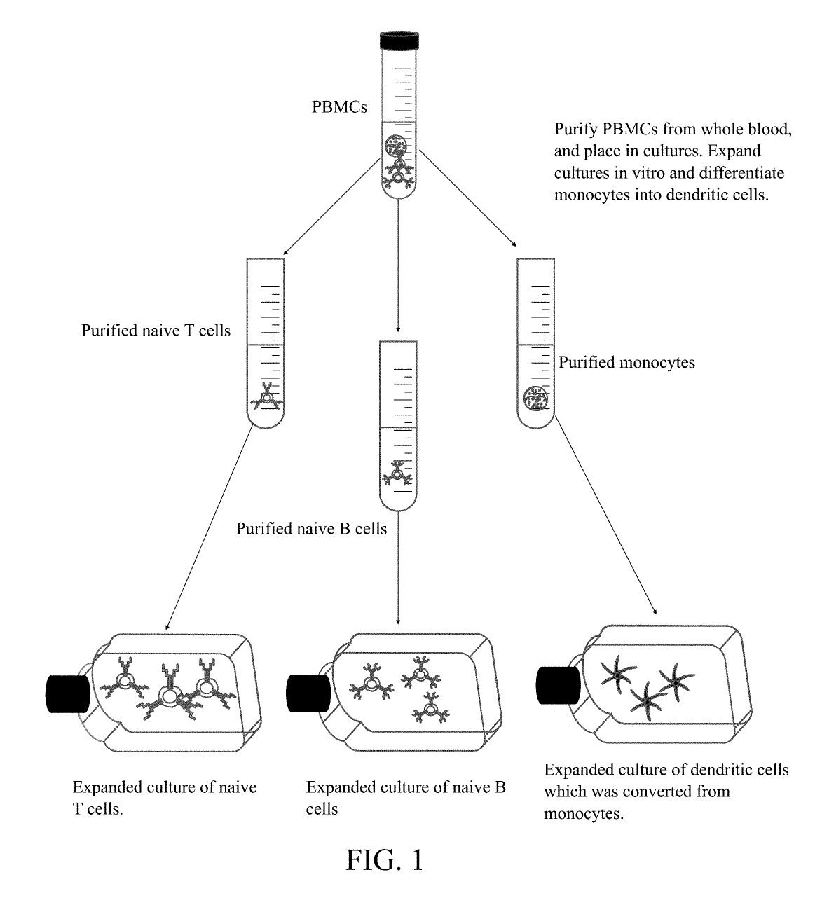

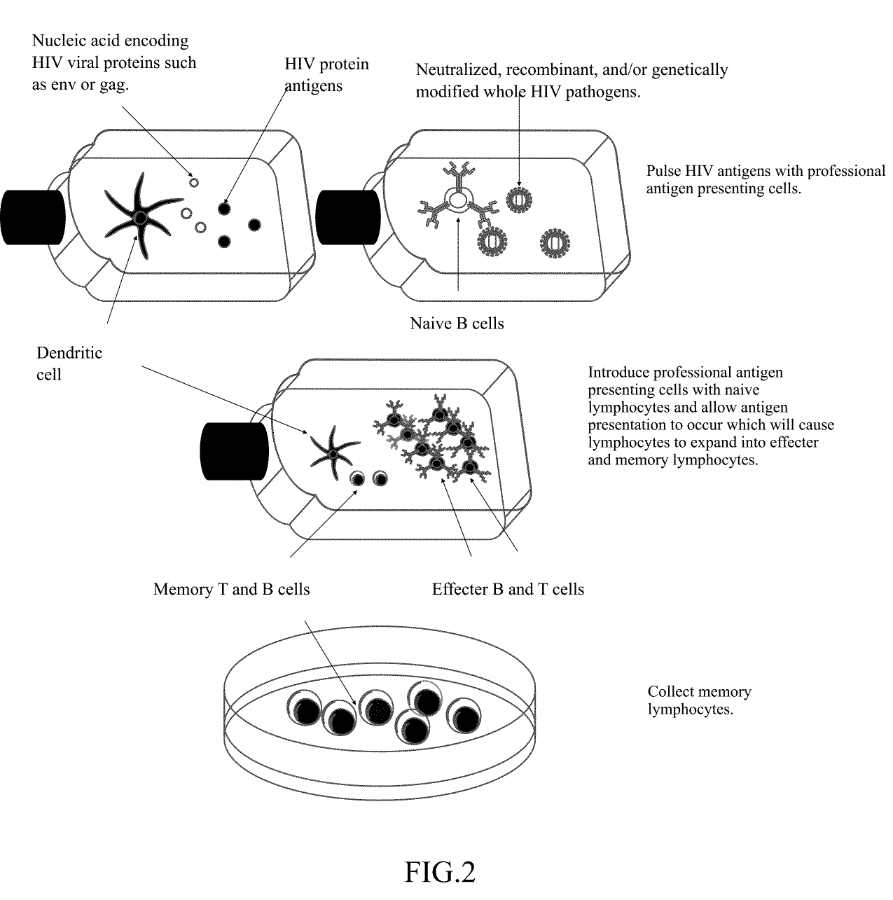

The invention is base in part, on a new method for providing immunological memory against various strains/clades of HIV “Immunological memory is sustained by long-lived antigen-specific lymphocytes that were induced by the original exposure, and that persist until a second encounter with the pathogen” (Janeway C A Jr, Travers P, Walport M, et al., 2001, para. 4). As shown in FIGS. 1 and 2, lymphocytes which were obtained from patient's immune system are cultured and stimulated in vitro with HIV antigens until a population of HIV antigen-specific memory lymphocytes is obtained. Creating memory lymphocytes against said pathogen in vitro instead of in vivo offers a direct approach to obtaining an appropriate amount of memory lymphocytes with a memory for different antigenic regions/strains of HIV. HIV have a high mutation rate, reproduces rapidly, and quickly overwhelm the immune system. In addition, the antibody response against HIV arrives days to weeks after initial infection and is non-neutralizing. “The initial neutralizing antibody response to this virus arises approximately 3 months after transmission and is strain-specific” (Liao, Hua-Xin et al., 2013, para 3). 1g141 antibodies to Gag p24 are first to appear followed by IgG1 antibodies to gp120 and gp41 Env proteins of relatively low affinity.

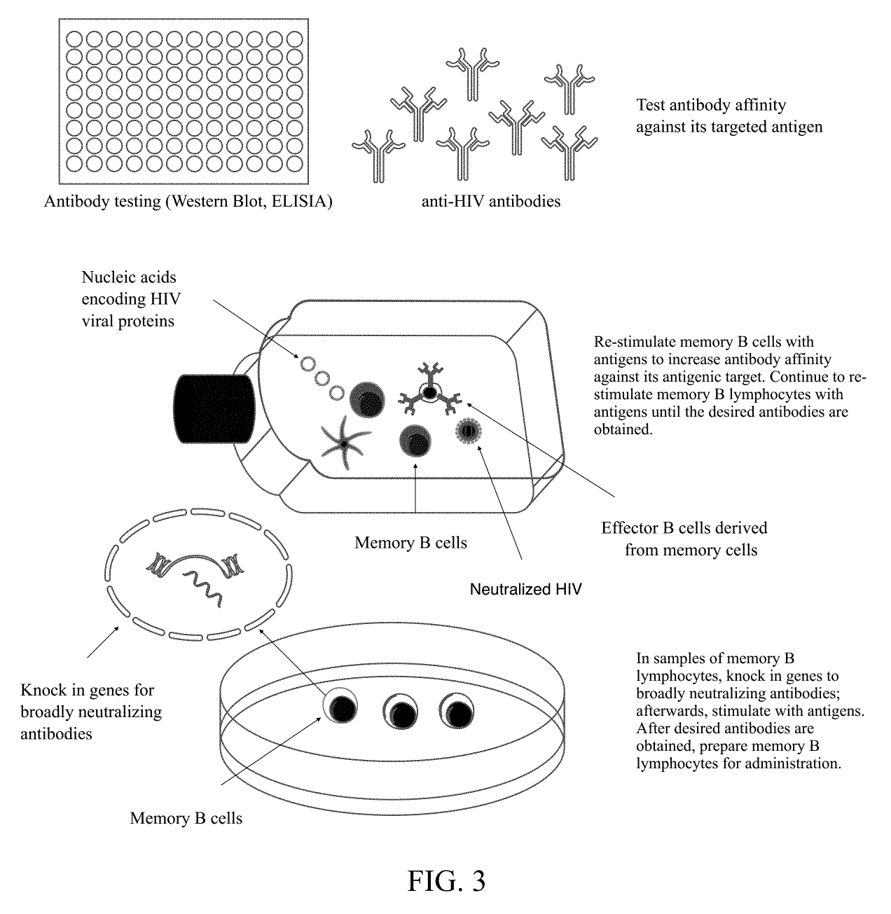

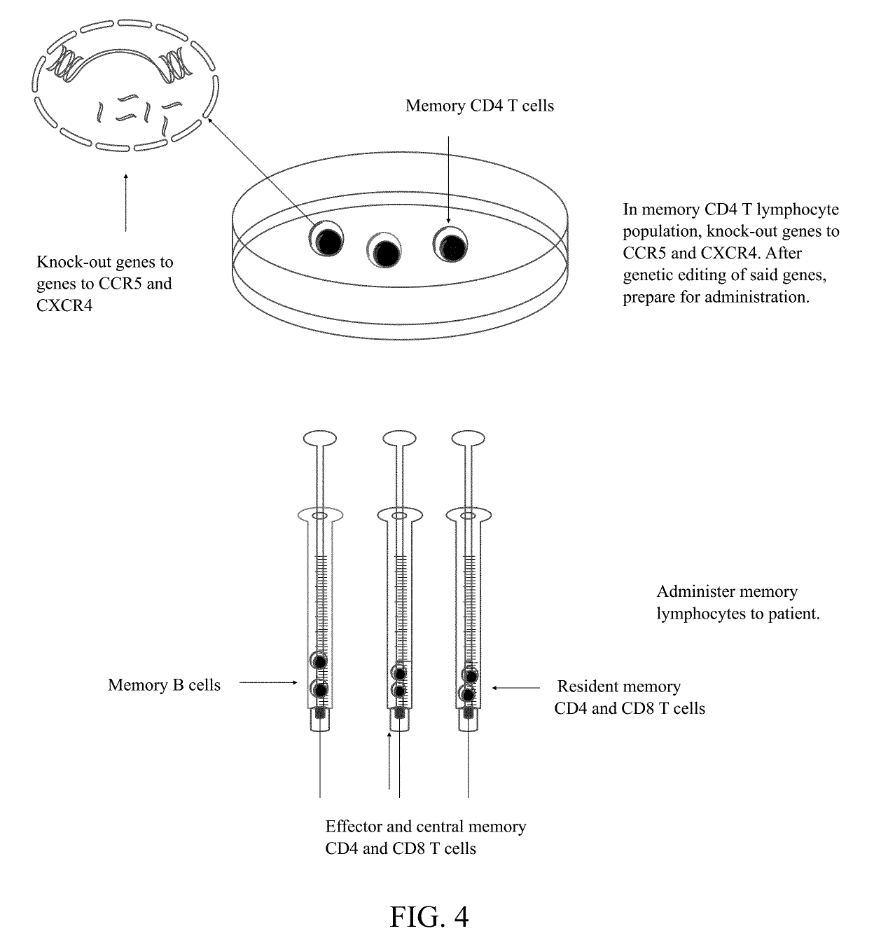

The invention provides a subject HIV antigen-specific memory B lymphocytes that have been educated to illicit broadly neutralizing HIV antibodies, neutralizing HIV antibodies, and/or non-neutralizing HIV antibodies upon a natural infection with HIV. “Memory B cells are long-lived descendants of cells that were once stimulated by antigen and had proliferated in the germinal center. These cells divide very slowly if at all; they express surface immunoglobulin but do not secrete antibody at a high rate” (Janeway C A Jr, Travers P, Walport M, et al., 2001, para. 9.9). As shown in FIGS. 1 & 2, memory B cells are created in vitro to have a memory against HIV antigens. However, memory B cells must have the ability to elicit broadly neutralizing antibodies upon an initial infection with HIV. Broadly neutralizing antibodies are monoclonal antibodies (mAbs) which have the ability to neutralize various strains of HIV. “Such mAbs include 2G12 (binds to mannose residues on gp120); b12 and F105 (bind to the CD4 binding site, CD4bs); 17b and ×5 (recognize conformational epitopes in the CD4i region); and 4E10 and 2F5 (bind to epitopes in the membrane proximal extracellular region or MPER of gp41)” (Balla-Jhagjhoorsingh, Sunita S. et al, 2016 para.1). As shown in FIGS. 3 and 4, antigen-specific memory B lymphocytes undergo a series of processes to obtain memory B cells which can illicit desired antibodies. One such process is to re-stimulate memory B lymphocytes repeatedly with said antigens to increase antibody affinity against its antigenic region of Env. Said lymphocytes can be re-stimulated with the same antigen to create neutralizing antibodies against its targeted epitope or differentlmutated antigenic strains to promote the production of broadly neutralizing antibody. Another process illustrated in FIG. 3, is the genetic modification in the variable regions of the light and heavy chain, wherein genes encoding variable regions of light and heavy chains for broadly neutralizing antibodies are knocked-in memory B lymphocytes which can recognize Env. Samples of memory B cells are isolated and genetically modified to encode genes for broadly neutralizing antibodies which includes, but is not limited to, VRC01, VRC02, VRC03, VRP04 or PGV04, VRC-PG0413, VRE-CH31, VRC-CH30, VRC-CH32, VRC-CH33, VRC-CH34, 10E8, PG9, P616, 3BNC117, NIH45-46, 12A12, 8ANC195, 3BNC60, 12A21, PGT121, PGT122, PGT123, PGT127, PGT128, PGT141, PGT142, PGT143, and PGT145, which are given in SEQ. NO. 1-56. In one ere bodiment, a sample of memory B cells which were created in vitro and have an affinity for Env are isolated, wherein it receives a broadly neutralizing antibody gene knocked in its variable light and heavy chain regions. In preferred embodiments, a minimum of 5 samples of memory B cells vhich were created in vitro and have an affinity for an epitope of Env is isolated for the genetic knock-in of a broadly neutralizing antibody which can target the same epitope of ENV. For example, one sample of memory B cells have genes for VRC01 given in SEQ. ID NO. 1-2 knocked in its variable heavy and light chain regions, and another sample has genes for 10E8 shown in SEQ. ID NO 21-22 knocked in said regions, and so on. B cells that remain from the main population receive repeated re-stimulation with HIV antigens as discussed earlier. “Although HIV-1 can escape from antibody monotherapy, combinations of broadly neutralizing antibodies (bNAbs) can effectively control HIV-1 infection and suppress viral load to levels below detection” (Klein et. al., 2012, para. 1). Although broadly neutralizing antibodies is the focus for antibody production through genetic modifications, it is not limited to abzymes, strain-specific neutralizing antibodies, or non-neutralizing antibodies.

As shown in FIG. 4, memory CD4 T (not naive or effector) cells undergo genetic modifications to certain chemokine receptors. These receptors include CCR5, CXCR4, and CCR8. Modification, wherein said genes are knocked out of memory T cells. Said receptors are required for HIV to infect cells, and knocking out genes associated to said receptors increases the survivability of these memory cells to survive long enough to differentiate from memory to effector helper T cells upon an initial infection.

In embodiments, the solution containing HIV antigen-specific immune memory lymphocytes are administered to patients without formaldehyde, thimerosal, or aluminum. There is a growing movement from concerned citizens across the world against vaccines. Citizens of this movement reject all forms of vaccination for themselves and for their children due to chemicals such as aluminum, formaldehyde, and thimerosal which are common in most vaccines. Said chemicals are not required in said solution in which the patient's HIV memory lymphocytes are suspended, and therefore will not be utilized. Instead, memory lymphocytes are suspended in blood plasma derived from said patient. Blood plasma can be obtained prior to administration or collected and stored during blood isolation.

### Operation

### Patient Examination

Patients would undergo a physical examine to determine the overall health of the patient. See, for example, meddean.luc.edu/lumen/meded/ipm Ipm2BSEBSE_laminated%20card.pdf, for details on physical exam procedure protocols. Said patient height, weight, age, sex, gender, race, current medication, and medical history should be noted. For example, a patient's weight will play a role in the amount of memory lymphocytes which can be administered, or for example, said patient age must be at an age when the immune system is fully functional. STIs, sepsis, leukemia, or cancers that are currently being treated with radiation can halt the progression of vaccine procedures, and must be noted in the exam. If said patient is HIV positive, then vaccine consideration would be halted, and treatment will be recommended. In addition, said patient must be informed of genetic modification procedures to memory lymphocytes created in vitro.

### Blood Extraction

Many methods of collecting blood from a subject are known to those in the skill of the art. See, for example, who.int/injection_safety/phleb_final_screen_ready.pdf for detailed information on phlebotomy procedures and safety protocols for the collection of whole blood. In preferred embodiments, whole blood is collected using a 16G needle in a 450-500 ml bag without filtration. The bag may contain chemicals such as sodium citrate, phosphate, and dextrose to prevent clotting during storage and transport. Additional chemicals can be added as needed such as adenine. Blood products can be shipped from a clinical site to a vaccine manufacturing company. Motion can be the motion associated with shipping. In another embodiment, cells can be gently rocked or rotated during incubation.

In yet another embodiment, whole blood can be collected using the venipuncture method with preferably a 21G butterfly needle, preferably in 10 ml tubes.Venipuncture can be applied when extra blood is needed in vaccine production or if certain blood cells are to be collected at a later date such as naive B lymphocytes or naive CD8 T lymphocytes.

### Cell Purification

Methods of isolating PBMCs from peripheral blood are known to those of skill in the art. Isolation can be done through density centrifugation and/or magnetic cell isolation, or through FACS (fluorescence-activated cell sorting). In preferred embodiments, magnetic cell isolation kits are used to collect PBMCs directly from whole blood. See, for example, www.stemcell.com/products/brands/rosettesep.html for products and directions on isolation procedures. PBMCs that are to be isolated are monocytes, naive T lymphocytes (CD4s and CD8s) and naive B lymphocytes. Methods of cell sorting such as FACS (fluorescence-activated cell sorting) is required to further isolate naive B lymphocytes expressing BCR receptors which can recognize Env or Gag or naive T cells with TCR receptors that can recognize different epitopes or polypeptides of HIV such as Env, Gag, etc. Monocytes can be cultured in eight welled suspension flask or in 100 ml flask containing mammalian cell culture medium and penicillin if needed. Naive CD4 T lymphocytes, Naive CD8 T lymphocytes, and Naive B lymphocytes, can be cultured in 300 ml suspension flask containing RPMI 1640 medium supplemented with 10% fetal calf serum (FCS), 1% sodium-pyruvate, 1% L-glutamine, 1% Penicillin/Streptomycin (10,000 U/ml/10,000 mg/ml), and 2% HEPES (1M, pH7.3) and optional leukemia inhibitor factors. Naive T lymphocytes are to be cultured in 3 .mu.g/ml phytohemagglutinin for 3 days and expanded with 5-20 U/ml recombinant interleukin-2 (rIL-2) for 5-6 days or until a minimum 0.5 million cells/ml in a minimum volume of 300 ml with the flask cap loosened to allow diffusion of oxygen and carbon dioxide. Naive B lymphocytes are to be cultured in 5-20 U/ml recombinant interleukin-2 (rIL-2) in the presence of recombinant CD40 ligand (rCD40L) for 5-6 days or until a minimum of 0.5 million cells/ml in a minimum volume of 300 ml with the flask cap loosened to allow diffusion of oxygen and carbon dioxide. In preferred embodiments, naive B lymphocytes that were isolated by FACs which contain BCR receptors for particular antigen (such as Env or Gag) are cultured in the same flask with naive CD4 T lymphocytes with TCR receptors that have the ability to recognize the same antigen upon stimulation. In another preferred embodiment, naive CD4 T lymphocytes are cultured in the same flask with naive CD8 T lymphocytes that have matching TCR receptors for any given antigenic epitope of HIV such as Gag, Env, etc. Examine each flask under a microscope and check for signs of abnormalities. Place samples in a humidified incubator set at 37 degrees C. and 5% CO. sub 2. Monitor cultures daily and change medium when needed; preferably, every 2-3 days.

### Monocyte Differentiation

Methods of monocyte differentiation are known to those of skill in the art. Enriched monocytes are differentiated into dendritic cells by culture in the presence of GM-CSF and IL-4 (see, e.g., U.S. Pat. No. 7,030,228). Commercial kits can be used for differentiation and expansion of macrophages and dendritic cells from monocytes.

In one embodiment, monocytes are differentiated into dendritic cells by culture in medium comprising a composition that induces differentiation of monocytes into dendritic cells. Suitable media for the culture of monocytes, immature and mature dendritic cells includes, but is not limited to, AIM-V, X-VIVO-15, RPMI and the like. Compositions that induce the differentiation of monocytes into dendritic cells are known to those of skill in the art, and includes but are not limited to, GM-CSF plus IL-4; GM-CSF plus IL-13. Enriched monocytes, are cultured in AIM-V medium, X-VIVO 15 medium, or other suitable medium in the presence 800 U/ml GM-CSF and 500 U/ml IL-4 for approximately 5-7 days, preferably six days in a humidified incubator set at 37.degree. C., 5% CO.sub.2 to allow the differentiation of monocytes into immature dendritic cells. Cytokine concentrations can be varied. In an alternative embodiment, monocytes are differentiated into macrophages comprising a composition that induces differentiation of monocytes into macrophages. Composition that induces the differentiation of monocytes into macrophages are known to those of skill in the art, and include, but are not limited to, M-CSF plus IL-4. Enriched monocytes, are cultured in AIM V medium, X-VIVO 15 medium, or other suitable medium in the presence 800 U/ml M-CSF and 500 U/ml IL-4 for approximately 4-7 days, preferably 6 days in a humidified incubator set at 37.degree. C., 5% CO.sub.2 to allow the differentiation of monocytes to macrophages. Cytokine concentration can be varied.

### Antigen Loading

Methods of loading APCs with antigens are known to those of skill in the art. After four-five days examine cultures and replace medium if needed. Once the vitality of the cultures have been established, pulse HIV viral antigens to professional antigen presenting cells (Dendritic Cells, Macrophages, and B lymphocytes). The APCs will then process said antigens and present said antigens on its cell surface in association with MHC molecules. Mix cells and medium in which they are suspended in with a pipet. Return the cultures to an incubator. Incubate cells for 12-24 hours at 37 degrees C. HIV antigens can be pulsed with any professional antigen presenting cells (Dendritic cells, Macrophages, and/or B lymphocytes); however, the preferred antigen presenting cells (APCs) are immature or mature dendritic cells. The antigen will then be processed and presented on the major histocompatibility complexes I or II. Examples of dendritic cell and macrophage antigens include, but are not limited to, HIV viral particles, proteins, epitopes, polypeptides, and nucleic acids. Antigens can be naturally occurring, chemically processed or recombinantly produced. Dendritic cells may be loaded with one or more HIV antigens as immature dendritic cells, mature dendritic cells, or during differentiation from immature to mature dendritic cells using methods known in the art. Accordingly, antigen loading can be performed simply by contacting the dendritic cell (pulsed) with the HIV antigen or nucleic acid encoding HIV antigen. One or more HIV antigens may be loaded directly into the dendritic cells, or nucleic acids encoding one or more HIV antigens may be loaded into the dendritic cells. In a preferred embodiment, the dendritic cells are pulsed with HIV antigens.

In one embodiment, antigens/pathogens can be loaded to naive B lymphocytes. Methods of loading B lymphocytes are known to those of skill in the art. Antigens include, but are not limited to, whole killed HIV pathogens (especially strains that are common to a region), whole killed genetically modified HIV antigens, or HIV viral particles, see for example, U.S. Pat. No. 7,608,273 or U.S. Pat. No. 7,067,134. One or more killed and/or modified HIV strains may be loaded directly into naive B lymphocytes. In one embodiment, naive B lymphocytes are loaded with strains of HIV antigens/pathogens which are most prevalent in natural infections. Said strains may be genetically modified to prevent infections and to enhance an immune response. In embodiments, if B cells are introduced to an attenuated pathogen, then rCD40 ligand must be present in the absence of T cells. If T cells are present, then prior to pathogen introduction, T cells must have undergone genetic modifications in their CCR5 and CXCR4 chemokine receptors to avoid infection, and they cannot be reintroduced to the patient.

### Antigen Presentation and Cellular Expansion

Methods for performing antigen presentation are known to those of skill in the art. Professional APCs which have undergone phagocytosis and display's loaded MHC I or II molecules with HIV antigens are to be diluted with naive lymphocytes. In one embodiment, loaded dendritic cells are diluted in flasks containing naive T lymphocytes (CD4 and CD8) preferably on a 1:1 ratio. In another embodiment, dendritic cells are diluted in flasks containing naive B and T lymphocytes, preferably on a 1:1 ratio. In yet another embodiment, loaded B lymphocytes act as the APC to present to naive CD4 T lymphocytes. Cytokines such as rIL 10 or rIL 15 can be added to culture medium to promote differentiation of naive lymphocytes to memory lymphocytes after stimulation. Incubate cultures for seven through fourteen days preferably in a system that promote cellular expansion such as Xuri Cell Expansion System W25, see, for example, promo.gelifesciences.com/GLAURF expansion.html#.WF5WGqOZM0o for detail description and directions for said device. Monitor cultures daily and check for signs of cell growth, viability, and cytokine production such as IL2.

### Collecting Memory Cells

Collect and isolate memory T and B lymphocytes from cultures or cellular expansion systems. A variety of methods is available to collect and isolate T and B lymphocytes from culture by those skilled in the art. Centrifuge sample for 5 minutes at 200 Gs. Collect supernatant and store in tubes; afterwards, collect cell pellet and place memory lymphocytes into a prepared flask according to their antigenic target and species. In one embodiment, FACs is used to further isolate memory lymphocytes according to their species and antigenic targets. For example, memory B lymphocytes whose receptors have a match for an antigenic portion of Env are isolated from memory CD4 T lymphocytes with the same or different antigenic targets, or from other memory B lymphocytes with different antigenic targets. Examine cultures under a microscope and check for signs of abnormalities. Afterwards, place cultures in a humidified incubator set at 37 degrees C.

### Antibody Testing

A variety of techniques are available in the art of testing antibody affinity and neutralizing capabilities against HIV antigens and are known to those of skill in the art. Techniques include but are not limited to Enzyme Immunoassays such as Elisa (Enzyme-linked Immunosorbent Assay) or Western Blotting. In preferred embodiments, antibody affinity against said antigens is tested through western blotting after a gel electrophoresis of the sample. See, for example, www.ncbi.nlm.nih.gov/pmc/articles/PMC3456489/ for a detailed description of western blotting procedures. Test supernatant which contained B lymphocytes from prior cellular expansion for broadly neutralizing, neutralizing and non-neutralizing HIV antibodies, and determine said antibody affinity for their targeted antigen determinant.

### Memory T Lymphocyte Genetic Modification

A variety of methods for editing genes is known to those of skill in the art. Methods for gene editing includes but is not limited to ZNF, TALEN, or CRISPR. Genetic alterations can be performed to deactivate or knock out genes to CCR5 in cultured memory CD4 T lymphocytes. In one embodiment commercial kits such as CRISPR/CAS (Clustered Regularly Interspaced Short Palindromic Repeats) which utilizes a homology direct repair pathway to knock genes in or out of cells can be used to knock-out said gene in memory CD4 T lymphocytes, see, for example, origene.com/search/ProductList.aspx?keyword=CCR5&go.x=0&go.y=0 for available kits and directions on the process of editing said gene. In another embodiment, commercial kits such as CRISPR/CAS will be used to knock-out genes to CXCR4 in said lymphocytes. After genetic modifications, place said lymphocytes in an incubator and incubate for 20 days or until administered to the subject. Memory B lymphocyte training and genetic modification

If neutralizing and/or broadly neutralizing antibodies were not created by B lymphocytes in vitro, then place memory B lymphocytes in culture and re-stimulate them with HIV antigens in the presence of armed helper T lymphocytes and/or recombinant CD40 ligand. See for example U.S. Pat. No. 8,840,890 on methods for re-stimulating memory B cells in vitro and coaching said B cell to produce desired antibody. In embodiments, memory B lymphocytes are re-stimulated with said antigens until neutralizing and/or broadly neutralizing antibodies have been detected in culture medium and displayed on the BCRs of memory B lymphocytes. Memory B lymphocytes can be re-stimulated with the same antigen and undergo further affinity maturation to increase its affinity and neutralizing capabilities against its targeted antigenic region such as epitopes of Env. Said lymphocytes can also be introduced to different strains of HIV or different antigenic regions to increase antibody affinity and broadly neutralizing antibody capabilities. Repeat process until B lymphocytes create desired antibodies.

In another embodiment, genes encoding broadly neutralizing antibodies are knocked-in to memory B lymphocytes genes which contain variable region coding sequences for light and/or heavy chains using commercial kits such as CRISPR see, for example, http://www.origene.com/search/ProductList.aspxkeyword=variable+region&go.x=0&go.y=0. Methods for knocking in genes are known to those of skill in the art, see, for example, U.S. Pat. No. 9,273,118. Examples of genes encoding broadly neutralizing antibodies are given in SEQ ID NO 1-56. Samples of memory B lymphocytes encoding ENV are to be isolated in new flasks containing fresh culture medium as discussed earlier. After isolation, knock-in genes for broadly neutralizing antibodies, for example, VRC01, 10E8, etc., using CRISPR/CAS, or other methods known to those of skill in the art. Knock-out genes in memory B lymphocytes immunoglobulin kappa locus (IGKV) variable region located on chromosomes 2 and/or immunoglobulin lambda locus (IGLU) variable region located on chromosome 22, and knock-in genes for variable light chains encoding light chains for broadly neutralizing antibodies. In addition knock-out genes in memory B-lymphocyte immunoglobulin heavy chain (IGHV) variable region located on chromosome 14, and knock in genes for variable region heavy chain for broadly neutralizing antibodies. Genes for broadly neutralizing antibodies are provided in the form of cDNA, see for example, origene.com/orf/?utm_source=Bing&utm_medium=cpc&utm_term=cDNA%2Bclone&utm_content=cDNA%2Bclones&utm campaign=Top%2BKeywords for products and details on cDNA. Once genetic modifications have been performed, re-stimulate said memory lymphocytes with antigens in the presence of helper T cells and/or recombinant CD40 ligand. Cytokines such as IL2 can be added to culture medium to stimulate proliferation. After seven-twenty days, isolate memory lymphocytes using FACS and check for BCRs indicating a similarity with the antibodies of interest. In addition, check culture medium using techniques discussed earlier for broadly neutralizing antibodies affinity against targeted antigen. Re-stimulate memory B lymphocytes with HIV antigens if necessary. Once desired antibodies are obtained, prepare memory B lymphocytes for administration to subject.

### Administration

Administration can be by methods known in the art to successfully deliver lymphocytes into contact with a subject's blood or near key organs/secondary organs of the lymphatic system such as the lymph nodes. Blood plasma is to be collected from the patient one-two days before administration. After collection, incubate blood plasma at 37 degrees C. Place memory lymphocytes in tubes with collected blood plasma and prepare for administration. Memory cells can be stored in a blood bank until administration. Memory cells can be delivered to a foreign recipient with matching histocompatibility proteins. Cells are administered in any suitable manner, often with pharmaceutically acceptable carriers. Suitable methods of administering cells in context of the present invention to a subject are available, and more than one route can be used to administer a particular cell composition. Routes of administration to said patient consist of intra-lymphatic, intravenous, and/or intra-dermal. In embodiments, HIV antigen-specific central memory CD4 and CD8 T cells, effector memory CD4 and CD8 T cells, and memory B cells can be administered intravenously and/or intra-lymphaticlly. In vivo, said lymphocytes migrate to secondary lymphoid organs, bone marrow, spleen, and/or circulates in the peripheral blood, lymphatic fluid, and interstitial fluid. Resident memory CD4 and CD8 T cells can be administered intramuscularly or intradermally near sites of natural infection such as the penis or vagina.

### Advantage

(a) Provides a direct method for establishing immunological memory with memory B cells which have the genetic capabilities for broadly neutralizing antibodies.
(b) Provides memory B cells which can secrete a small amount of neutralizing antibodies against HIV despite infection.
(c) Provides memory CD4 T cells with certain chemokine receptors knock-out of its genome. These memory T cells can resist entry from HIV to some extent upon infection.
(d) Provides a means of vaccination which do not contain chemicals that certain facets of the public oppose to.
(e) Provides a means to vaccinate patients who couldn't normally receive vaccination due to its immune response. Because all immune responses are conducted in vitro, patients such as pregnant women or chemotherapy patients can receive said vaccination without the risk of inflammation which can jeopardize their health.
(f) Provides a direct method for establishing an adequate amount of antigen-specific memory lymphocytes needed to provide an appropriate immune response against HIV.

### CONCLUSION

Accordingly, the reader will see that the vaccine closures of the various embodiments can be used to establish immunological memory against HIV with humoral immune cells which can elicit antibodies of high affinity against numerous strains/clades of HIV. In addition, because memory T cells have undergone gene editing to genes for CCR5 and CXCR4, said patient can have T cells that can survive infection long enough to orchestrate and activate the appropriate secondary response.

What is claimed is:

1. A method for providing immunological memory against different strains of HIV, comprising:(a) creating HIV antigen-specific adaptive immune memory lymphocytes in vitro from a subject naive T & B lymphocytes, and (b) administrating HIV antigen-specific adaptive immune memory lymphocytes from step (a) to said subject.

2. The method of claim 1, wherein the antigen-specific adaptive immune memory lymphocytes are HIV antigen-specific immune memory B lymphocytes.

3. The method of claim 2, wherein the antigen-specific adaptive immune memory B lymphocytes are genetically modified in variable light and heavy chain regions in vitro to contain genes for broadly neutralizing antibodies.

4. The method of claim 1, wherein the antigen-specific adaptive immune memory lymphocytes are HIV antigen-specific immune memory T lymphocytes.

5. The method of claim 4, wherein the antigen-specific adaptive immune memory CD4 T lymphocytes are genetically modified in vitro to have genes to CCRS and CXCR4 knocked out of said lymphocytes.

6. The method of claim 1, wherein said vaccine is administered before exposure to HIV.


SEQUENCE LISTING (57 sequences)

SEQ ID NO: 1 - DNA, 363 bp, Artificial Sequence
  cDNA/VRC01 anti-HIV-1 immunoglobulin heavy chain variable region
caggtgcagctggtgcagtctgggggtcagatgaagaagcctggcgagtcgatgagaatttcttgtcgggcttctggatatgaatttattgattgtacgctaaattggattcgtctggcccccggaaaaaggcctgagtggatgggatggctgaagcctcgggggggggccgtcaactacgcacgtccacttcagggcagagtgaccatgactcgagacgtttattccgacacagcctttttggagctgcgctcgttgacagtagacgacacggccgtctacttttgtactaggggaaaaaactgtgattacaattgggacttcgaacactggggccggggcaccccggtcatcgtctcatca

SEQ ID NO: 2 - DNA, 312 bp, Artificial Sequence
  cDNA/VRC01 anti-HIV-1 immunoglobulin light chain variable region
gaaattgtgttgacacagtctccaggcaccctgtctttgtctccaggggaaacagccatcatctcttgtcggaccagtcagtatggttccttagcctggtatcaacagaggcccggccaggcccccaggctcgtcatctattcgggctctactcgggccgctggcatcccagacaggttcagcggcagtcggtgggggccagactacaatctcaccatcagcaacctggagtcgggagattttggtgtttattattgccagcagtatgaattttttggccaggggaccaaggtccaggtcgacattaagcga

SEQ ID NO: 3 - DNA, 363 bp, Artificial Sequence
  cDNA/VRC02 anti-HIV-1 immunoglobulin heavy chain variable region
caggtgcagctggtgcagtctgggggccagatgaagaagcctggcgagtcgatgagaatttcttgtcaggcttccggatatgaatttattgattgtacactaaattgggttcgcctggcccccggaagaaggcctgaatggatgggatggctgaagcctcgagggggggccgtcaactacgcacgtccacttcaaggcagagtgaccatgactcgagacgtgtattccgacacagcctttttggagctgcgctccttgacagcagacgacacggccgtctactattgtactaggggaaaaaattgtgattacaattgggacttcgaacactggggccggggtaccccggtcaccgtctcatca

SEQ ID NO: 4 - DNA, 312 bp, Artificial Sequence
  cDNA/VRC02 anti-HIV-1 immunoglobulin light chain variable region
gaaattgtgttgacacagtctccaggcaccctgtctttgtctccaggggaaacagccatcatctcttgtcggaccagtcagtatggttccttagcctggtatcaacagaggcccggccaggccccccggctcgtcatctattcgggctctactcgggccgcaggcatcccagacaggttcagcggcagtcggtgggggccagactacaatctcaccatcaggaacctggagtcgggagattttggtctttattattgccagcagtatgaattttttggccaggggaccaaggtccaggtcgacattaagcga

SEQ ID NO: 5 - DNA, 390 bp, Artificial Sequence
  cDNA/VRC03 anti-HIV-1 immunoglobulin heavy chain variable region
caggtgcagctggtgcagtctggggctgtgattaagacgcctgggtcctcagtgaagatctcatgtcgggcttctggatacaactttcgtgattattcgatccattgggtccgcctcattcctgacaagggatttgagtggattggatggattaaacctctgtggggtgccgtcagttatgcccggcaacttcagggccgagtctctatgactcgacaattatctcaagacccagacgacccggactggggcgttgcctacatggagttcagtggactgacgcccgccgacacggccgaatatttttgtgtccggagagggtcctgtgattattgcggagactttccctggcaatactggggtcagggcaccgtcgtcgtcgtctcgtca

SEQ ID NO: 6 - DNA, 309 bp, Artificial Sequence
  cDNA/VRC03 anti-HIV-1 immunoglobulin light chain variable region
gaaattgtgttgacgcagtctcccggcatcctgtctctgtctccaggagagacagccaccctcttttgtaaggccagtcagggtggcaatgctatgacgtggtatcagaagagacgtggccaggttcccagactcctgatctacgatacatctcgcagggcctctggcgttcctgacagatttgttggcagtgggtctgggacagacttctttctcacgatcaacaaattggaccgggaagatttcgcagtctattattgtcaacaatttgaattttttggcctggggagcgagctggaagtccatcga

SEQ ID NO: 7 - DNA, 375 bp, Artificial Sequence
  cDNA/VRC-PG04 anti-HIV-1 immunolobulin heavy chain variable region
caggtgcagctggtgcagtctgggtctggagtgaagaagcctggggcttcggtgagagtctcctgttggacctctgaggacatcttcgaaagaaccgagttgattcattgggtgcgacaggcccctggacaaggacttgagtggattgggtgggtgaaaaccgtcactggcgccgtcaattttggttcaccagattttcgacagagagtctctctgacccgcgacagggacctcttcacagcccatatggacattcgcggactgacacaaggcgacacggccacatatttttgcgcgagacagaaattttatacgggcggccaaggctggtacttcgatctctggggccgtggaaccctcattgttgtctcgtca

SEQ ID NO: 8 - DNA, 306 bp, Artificial Sequence
  cDNA/VRC-PG04 anti-HIV-1 immunoglobulin light chain variable region
gaaattgtgttgacgcagtctccaggcaccctgtctttgtctccaggggaaacagccagcctctcctgcacggcggcctcatatggtcacatgacttggtatcagaaaaaacctggccagcctcccaaactcctcatttttgcaacgtccaagagggccagtggcatcccagacaggttcagtggctcccagtttggtaaacaatatactctcaccatcaccagaatggagcctgaagattttgcgagatattactgtcagcaacttgaattcttcggccaggggacacgactggagattagacga

SEQ ID NO: 9 - DNA, 375 bp, Artificial Sequence
  cDNA/VRCPG04b anti-HIV-1 immunoglobulin heavy chain variable region
caggtgcagctggtgcagtctgggtctggagtgaagaagcctggggcttcggtgagagtctcctgttggacctctgaggacatcttcgaaagaaccgaattgattcattgggtgcgacaggcccctggacaaggacttgagtggattgggtgggtgaaaactgtcactggcgccgtcaattttggttcaccaaattttcgacatagagtctctctgacccgcgacagggacctcttcacagcccatatggacattcgcggactgacacaaggcgacacggccacatatttttgcgcgagacagaaatttgagaggggcggccaaggctggtatttcgatctctggggccgtggaaccctcattgttgtctcgtca

SEQ ID NO: 10 - DNA, 303 bp, Artificial Sequence
  cDNA/VRC-PG04b anti-HIV-1 immunoglobulin light chain variable region
gaaattgtgttgacgcagtctccaggcaccctgtctttgtctccaggggaaacagccagcctctcctgcacggcggcctcatatggtcacatgacttggtatcagaaaaaacctggccagcctcccaaactcctcatttttgcaacgtccaagagggccagtggcatcccagacaggttcagtggctcccagtttggtaaacaatacactctcaccatcaccagaatggagcctgaagattttgcgggatattactgtcagcaagttgaattcttcggccaggggacacgactggagattaga

SEQ ID NO: 11 - DNA, 393 bp, Artificial Sequence
  cDNA/VRC-CH31 anti-HIV-1 immunoglobulin heavy chain variable region
caggtgcagctggtgcagtcaggggctgccgtgaggaagcctggggcctcagtgactgtctcctgtaaattcgctgaagacgacgactactctccatactgggtgaatccggcccctgaacattttattcactttttgcgacaggcccctggacaacaactagagtggctggcatggatgaacccaacgaatggcgccgttaattatgcatggtaccttaatggcagggtcacggcgaccagggacaggtccatgactacagcctttttggaagtgaagagtctaagatctgacgacacggccgtctactattgtgcgagggcccagaaaagggggcggagtgagtgggcctacgcccactggggtcagggcactccggtcgtcgtctcgtca

SEQ ID NO: 12 - DNA, 309 bp, Artificial Sequence
  cDNA/VRC-CH31 anti-HIV-1 immunoglobulin light chain variable region
gacatccagatgacccagtctccatcgtccctgtctgcatcactcggggacagagtcaccatcacttgccaggcgagtcggggcattggcaaagatttaaattggtaccagcagaaagcgggaaaagcccctaagttactggtctctgatgcatccactttggaagggggggtcccatcaaggttcagtgggagtggatttcaccaaaattttagtctgactatcagcagcctgcaggctgaggatgttgcaacatacttctgtcaacaatacgagacttttggccaggggaccaaggtggacatcaaa

SEQ ID NO: 13 - DNA, 393 bp, Artificial Sequence
  cDNA/VRC-CH30 anti-HIV-1 immunoglobulin light chain variable region
caggtgcagctggtgcagtcaggggctgccgtgaggaagcctggggcctcagtgactgtctcctgcaaattcgctgaagacgacgactactctccacactgggtgaatccggcccctgaacactatattcactttctacgacaggcccctggacagcaactggagtggttggcatggatgaaccctacgaatggcgccgtcaattatgcatggcagcttcatggcaggctcacggcgaccagagacgggtccatgactacagcctttttggaagtgaggagtctaagatctgacgacacggccgtctattattgtgcgagggcccagaaaagggggcggagtgaatgggcctacgcccactggggccagggaaccccggtcgccgtctcctca

SEQ ID NO: 14 - DNA, 309 bp, Artificial Sequence
  cDNA/VRC-CH30 anti-HIV-1 immunoglobulin light chain variable region
gacatccagatgacccagtctccatcgtccttgtctgcatcactcggagacagagtcaccatcacttgtcaggcgagtcggggcattggtaaagatttaaattggtaccagcagaaaccgggaaaggcccctaagttactggtctctgatgcatccattttggaagggggggtcccatcaaggttcagtgggagtggatttcaccaaaattttagtctgaccatcagcagcctgcagcctgaggatgttgcgacatacttctgtcagcagtacgagacttttggccaggggaccaaagtggacatcaaa

SEQ ID NO: 15 - DNA, 393 bp, Artificial Sequence
  cDNA/VRC-CH32 anti-HIV-1 immunoglobulin heavy chain variable region
caggtgcagctggtgcagtcaggggctgccgtgaggaagcctggggcctcagtgactgtctcctgcaagttcgctgaagacgacgacttctctccacactgggtgaatccggcccctgaacactatattcattttctgcgacaggcacctggacaacaactagagtggttggcatggatgaagcctacgaatggtgccgtcaattatgcatggcaacttcagggcagggtcacggtgaccagggacaggtcccagactacagcctttttggaagttaagaatctgagatctgacgacacggccgtctattattgtgcgagggcccagaaaagggggcgcagcgagtgggcctatgcccactggggccagggaaccccggtcgtcatctccgca

SEQ ID NO: 16 - DNA, 309 bp, Artificial Sequence
  cDNA/VRC-CH32 anti-HIV-1 immunoglobulin light chain variable region
gacatccagatgacccagtctccatcgtccctgtctgcatcactcggagacagagtcaccatcacttgccaggcgagtcggggcattggcaaagatttaaattggtaccaacagaaaccgggaagagcccctaagttactagtctctgatgcatccattttggaagggggggtcccaacgagattcagtgggagtggatttcaccaaaactttagcctgaccatcagcagcctgcaggctgaggatgttgcaacatatttctgtcagcaatacgaaacttttggccaggggaccaaggtggacatcaaa

SEQ ID NO: 17 - DNA, 393 bp, Artificial Sequence
  cDNA/VRC-CH33 anti-HIV-1 immunoglobulin heavy chain variable region
caggtgcagctggtgcagtcaggggctgccgtgaggaagcccggggcctcaattagtgtctcctgcaaattcgctgatgccgacgactactctccgcactggatgaatccggcccctgaacactatattcactttctgcgccaggcccctggacagcaattagagtggttggcgtggatgaatcctacgaatggcgccgttaattatgcctggtaccttaatggcagggtcacggcgaccagagacaggtccatgaccacagcgtttctggaagtgaggagtctgagatctgacgacacggccgtctattattgtgcgagggcccagaaaagggcgcggagtgaatgggcctacgcccactggggccagggaaccccggtcgtcgtctcctca

SEQ ID NO: 18 - DNA, 309 bp, Artificial Sequence
  cDNA/VRC-CH33 anti-HIV-1 immunoglobulin light chain variable region
gacatccagatgacccagtctccatcgtccttgtctgcatcactcggagacagagtcaccatcacttgccaggcgagtcggggcattggtaaagatttaaattggtatcagcagaaacggggaagagcccctaggttactggtctctgatgcctccgttttggaagggggggtcccatcaaggttcagtggcagtggatttcaccaaaactttagcttgaccatcagcaccctgcagcctgaggatgttgcgacatacttctgtcagcagtacgagacttttggccaggggaccaaagtggacatcaaa

SEQ ID NO: 19 - DNA, 393 bp, Artificial Sequence
  cDNA/VRC-CH34 anti-HIV-1 immunoglobulin heavy chain variable region
caggtgcagctggtgcagtcgggggctgccgtgaggaagcctggggcctcagtaactgtctcctgcaaattcgctgaagacgacgactggtctccacactgggtgaatccggcccctgaacactatattcattttctacggcaggcccctggacagcaattagagtggttggcatggatgaaccctacaaatggtgccgtcaattatgcatggcagcttaacggcaggctcacggcgaccagagacacgtccatgactacagcctttttggaggtgaagagtctgagatctgacgacacggccgtctattattgtgcgagggcccaaaaaagggggcgcagtgaatgggcctacgcccactggggccagggaaccccggtcgtcgtctcctca

SEQ ID NO: 20 - DNA, 309 bp, Artificial Sequence
  cDNA/VRC-CH34 anti-HIV-1 immunoglobulin light chain variable region
gacatccagatgacccagtctccatcgtccttgtctgcatcactcggagacagagtcaccatcacttgccaggcgagccggggtattggtaaagatttaaattggtaccaacagaaagcgggaaaagcccctaagttactggtctctgatgcatccattttggaagggggggtcccatcaaggttcagcgggagtggatttcaccaaaattttagtctgaccatctccagcctgcagcctgaagatgttgcgacctatttctgtcagcagtacgagacttttggccaggggaccaaggtggacatcaaa

SEQ ID NO: 21 - DNA, 393 bp, Artificial Sequence
  cDNA/10E8 anti-HIV-1 immunoglobulin heavy chain variable region
gaggtgcagctggtggagtctgggggaggcttggtgaagcctggaggatcccttagactctcatgttcagcctctggtttcgacttcgataacgcctggatgacttgggtccgccagcctccagggaagggcctcgaatgggttggtcgtattacgggtccaggtgaaggttggtcagtggactatgctgcacccgtggaaggcagatttaccatctcgagactcaattcaataaatttcttatatttggagatgaacaatttaagaatggaagactcaggcctttacttctgtgcccgcacgggaaaatattatgatttttggagtggctatccgccgggagaagaatacttccaagactggggccggggcaccctggtcaccgtctcctca

SEQ ID NO: 22 - DNA, 328 bp, Artificial Sequence
  cDNA/10E8 anti-HIV-1 immunoglobulin light chain variable region
tcctatgagctgactcaggagactggtgtctctgtggccctgggacggacagtcacaatcacgtgccggggagacagcctcagaagtcattatgcaagttggtaccaaaagaagccaggacaggcccctatacttctcttctatggtaaaaataatcgtccttcaggggtcccagaccgattctctggctccgcctcaggaaacagagcttccttgaccatctctggggctcaggcggaagacgacgcggaatattattgtagttctcgggacaagagtggcagccgtctgtcggtcttcggcggggggaccaaactgaccgtcctca

SEQ ID NO: 23 - DNA, 408 bp, Artificial Sequence
  cDNA/PG9 anti-HIV-1 immunoglobulin heavy chain ariable region
cagcgattagtggagtctgggggaggcgtggtccagcctgggtcgtccctgagactctcctgtgcagcgtccggattcgacttcagtagacaaggcatgcactgggtccgccaggctccaggccaggggctggagtgggtggcatttattaaatatgatggaagtgagaaatatcatgctgactccgtatggggccgactcagcatctccagagacaattccaaggatacgctttatctccaaatgaatagcctgagagtcgaggacacggctacatatttttgtgtgagagaggctggtgggcccgactaccgtaatgggtacaactattacgatttctatgatggttattataactaccactatatggacgtctggggcaaagggaccacggtcaccgtctcgagc

SEQ ID NO: 24 - DNA, 330 bp, Artificial Sequence
  cDNA/PG9 anti-HIV-1 immunoglobulin light chain variable region
cagtctgccctgactcagcctgcctccgtgtctgggtctcctggacagtcgatcaccatctcctgcaatggaaccagcaatgatgttggtggctatgaatctgtctcctggtaccaacaacatcccggcaaagcccccaaagtcgtgatttatgatgtcagtaaacggccctcaggggtttctaatcgcttctctggctccaagtccggcaacacggcctccctgaccatctctgggctccaggctgaggacgagggtgactattactgcaagtctctgacaagcacgagacgtcgggttttcggcactgggaccaagctgaccgttcta

SEQ ID NO: 25 - DNA, 411 bp, Artificial DNA
  cDNA/PG16 anti-HIV-1 immunoglobulin heavy chain variable region
caggaacaactggtggagtctgggggaggcgtggtccagccgggggggtccctgagactctcctgtttagcgtctggattcacgtttcacaaatatggcatgcactgggtccgccaggctccaggcaagggcctggagtgggtggcactcatctcagatgacggaatgaggaaatatcattcagactccatgtggggccgagtcaccatctccagagacaattccaagaacactctttatctgcaattcagcagcctgaaagtcgaagacacggctatgttcttctgtgcgagagaggctggtgggccaatctggcatgacgacgtcaaatattacgattttaatgacggctactacaactaccactacatggacgtctggggcaaggggaccacggtcaccgtctcgagc

SEQ ID NO: 26 - DNA, 330 bp, Artificial Sequence
  cDNA/PG16 anti-HIV-1 immunoglobulin light chain variable region
cagtctgccctgactcagcctgcctccgtgtctgggtctcctggacagacgatcaccatctcctgcaatggaaccagcagtgacgttggtggatttgactctgtctcctggtaccaacaatccccagggaaagcccccaaagtcatggtttttgatgtcagtcatcggccctcaggtatctctaatcgcttctctggctccaagtccggcaacacggcctccctgaccatctctgggctccacattgaggacgagggcgattatttctgctcttcactgacagacagaagccatcgcatattcggcggcgggaccaaggtgaccgttcta

SEQ ID NO: 27 - DNA, 387 bp, Artificial Sequence
  cDNA/3BNC117 anti-HIV-1 immunoglobulin heavy chain variable region
caggtccaattgttacagtctggggcagcggtgacgaagcccggggcctcagtgagagtctcctgcgaggcttctggatacaacattcgtgactactttattcattggtggcgacaggccccaggacagggccttcagtgggtgggatggatcaatcctaagacaggtcagccaaacaatcctcgtcaatttcagggtagagtcagtctgactcgacacgcgtcgtgggactttgacacattttccttttacatggacctgaaggcactaagatcggacgacacggccgtttatttctgtgcgcgacagcgcagcgactattgggatttcgacgtctggggcagtggaacccaggtcactgtctcgtcagcgtcgaccaagggccca

SEQ ID NO: 28 - DNA, 315 bp, Artificial Sequence
  cDNA/3BNC117 anti-HIV-1 immunoglobulin kappa chain variable region
gacatccagatgacccagtctccatcctccctgtctgcctctgtgggagataccgtcactatcacttgccaggcaaacggctacttaaattggtatcaacagaggcgagggaaagccccaaaactcctgatctacgatgggtccaaattggaaagaggggtcccatcaaggttcagtggaagaagatgggggcaagaatataatctgaccatcaacaatctgcagcccgaagacattgcaacatatttttgtcaagtgtatgagtttgtcgtccctgggaccagactggatttgaaacgtacggtggctgcacca

SEQ ID NO: 29 - DNA, 398 bp, Artificial Sequence
  cDNA/NIH45-46 anti-HIV-1 immunoglobulin heavy chain variable region
caagtgcgactgtcgcagtctggaggtcagatgaagaagcctggcgagtcgatgagactttcctgtcgggcttccggatatgaatttctgaattgtccaataaattggattcgcctggcccccggaagacggcctgagtggatgggatggctgaagcctaggggaggggccgtcaattacgcacgtaaatttcagggcagagtgaccatgactcgagacgtgtattccgacacagcctttttggagttgcgctccttgacatcagacgacacggccgtctatttttgtactaggggaaaatattgtactgcgcgcgactattataattgggacttcgaacactggggccggggtgccccggtcaccgtctcatcagcgtcgaccaagggcccatcggt

SEQ ID NO: 30 - DNA, 327 bp, Artificial Sequence
  cDNA/NIH45-46 anti-HIV-1 immunoglobulin kappa chain variable region
gaaattgtgttgacacagtctccagccaccctgtctttgtctccaggggaaacagccatcatctcttgtcggaccagtcagtctggttccttagcctggtatcaacagaggcccggccaggcccccaggctcgtcatctattcgggttctactcgggccgctggcatcccagacaggttcagcggcagtcggtggggggcagactacaatctcagcatcagcaacctggagtcgggagattttggtgtttattattgtcagcagtatgaattttttggccaggggaccaaggtccaggtcgacatcaaacgtacggtggctgcacca

SEQ ID NO: 31 - DNA, 398 bp, Artificial Sequence; identical to SEQ ID NO: 29
  cDNA/12A12 anti-HIV-1 immunoglobulin heavy chain variable region

SEQ ID NO: 32 - DNA, 327 bp, Artificial Sequence; identical to SEQ ID NO: 30
  cDNA/12A12 anti-HIV-1 immunoglobulin kappa light chain variable region

SEQ ID NO: 33 - DNA, 423 bp, Artificial Sequence
  cDNA/8ANC195 anti-HIV immunoglobulin heavy chain variable region
cagatacacctcgtacaatctgggaccgaagtcaagaagcctgggtcctcggtgacggtctcctgcaaggcctatggagtcaacactttcggtctctatgccgtcaattgggtgcgacaggccccaggacaaagtcttgagtacataggacagatatggcggtggaaatccagcgcttcacatcatttccggggccgagtcctcatttccgcggtggacctcacgggctcctcgccgcctatttcttccttggagatcaagaacctgacctctgacgacacggccgtctatttttgcacgacaacctcgacgtacgacaagtggagtggccttcatcacgacggggtcatggcattttcctcttggggccagggaaccctcatctccgtctccgcggcgtcgaccaagggcccatcggtcttc

SEQ ID NO: 34 - DNA, 390 bp, Artificial Sequence
  cDNA/8ANC195 anti-HIV immunoglobulin kappa chain variable region
gacatccagatgacccagtctccttccaccctgtctgcatctataggtgacacagtcaggatctcttgccgggccagccagagtatcactggtaactgggtggcgtggtatcagcagagaccagggaaagcccctagactcctcatttataggggagccgcattactaggtggggtcccgtcaaggttcagcggcagtgcagctgggacagacttcactctcaccatcggcaacctgcaggctgaagattttggaactttttactgccaacagtatgacacttatccagggaccttcggccaagggactaaagttgaggtgaaacgtacggtggctgcaccatctgtcttcatcttcccgccatctgatgagcagttgaaatctggaact

SEQ ID NO: 35 - DNA, 391 bp, Artificial Sequence
  cDNA/3BNC60 anti-HIV-1 immunoglobulin heavy chain variable region
caggtccatttgtcacagtctggggcagcggtgacgaagcccggggcctcagtgagagtctcctgcgaggcttccggatacaagattagtgaccactttattcattggtggcgacaggccccaggacagggccttcagtgggtggggtggatcaatcctaagactggtcagccaaacaatcctcgtcaatttcagggtagagtcagtctgactcgacaggcgtcgtgggactttgacacatattccttttacatggacctcaaggcagtaagatcggacgacacggccatttatttctgtgcgcgacaacgcagcgacttttgggatttcgacgtctggggcagcggcacgcaggtcactgtctcgtcagcgtcgaccaagggcccatcgg

SEQ ID NO: 36 - DNA, 312 bp, Artificial Sequence
  cDNA/3BNC60 anti-HIV-1 immunoglobulin kappa chain variable region
gacatccagatgacccagtctccatcctccctgtctgcacgtgtaggcgataccgtcactatcacttgccaggcaaacggctacttaaattggtatcaacagagacgagggaaagccccaaaactcctgatctacgatgggtccaaattggagagaggcgtcccagcaaggttcagtggaagaagatggggacaagaatataatctgaccatcaacaatctgcagcccgaagacgttgcaacatatttttgtcaagtgtatgagtttatcgtccctgggaccagactggatttaaaacgtacggtggctgca

SEQ ID NO: 37 - DNA, 383 bp, Artificial Sequence
  cDNA/12A21 anti-HIV-1 immunoglobukin heavy chain region
tcccagcatttggtgcaatctgggactcaggtgaagaagcctggggcctcagtgcgggtctcctgccaggcttctggatataccttcaccaattacattctccactggtggcgacaggcccctggacaagggctggagtggatgggattgatcaagcctgtctttggtgccgtaaattacgcgcgccagtttcagggcaggattcagttgactagggacatctacagggagatagccttcctggacctgagtggcctcagatctgacgacacggccgtctattactgtgcgcgagatgagagcggggacgacctcaagtggcacctacatccctggggccagggaacgcaggtcatagtttccccagcgtcgaccaagggccc

SEQ ID NO: 38 - DNA, 315 bp, Artificial Sequence
  cDNA/12A21 anti-HIV-1 immunoglobulin kappa chain variable region
gacatccagatgacccagtctccatcctccctgtctgcatctgtaggagacagagtcaccataaactgccaggcgggtcagggcattggctcctctttaaattggtatcaaaaaaaaccagggagagcccctaagctcctggtccacggcgcttccaatcttcaaagaggggtcccatcgaggttcagtggaagtggatttcacacaactttcactctcaccatcagcagcctgcagcctgacgatgttgcgacatacttctgtgcggtctttcaatggttcggccctgggaccaaagtggatatcaaacgtacg

SEQ ID NO: 39 - DNA, 396 bp, Artificial Sequence
  cDNA/PGT121 anti-HIV-1 immunoglobulin heavy chain variable region
cagatgcagttacaggagtcgggccccggactggtgaagccttcggaaaccctgtccctcacgtgcagtgtgtctggtgcctccataagtgacagttactggagctggatccggcggtccccagggaagggacttgagtggattgggtatgtccacaaaagcggcgacacaaattacagcccctccctcaagagtcgagtcaacttgtcgttagacacgtccaaaaatcaggtgtccctgagccttgtggccgcgaccgctgcggactcgggcaaatattattgcgcgagaacactgcacgggaggagaatttatggaatcgttgccttcaatgagtggttcacctacttctacatggacgtctggggcaatgggactcaggtcaccgtctcctca

SEQ ID NO: 40 - DNA, 315 bp, Artificial Sequence
  cDNA/PGT121 anti-HIV-1 immunoglobulin light chain variable region
tccgatatatctgtggccccaggagagacggccaggatttcctgtggggaaaagagccttggaagtagagctgtacaatggtatcaacacagggccggccaggccccctctttaatcatatataataatcaggaccggccctcagggatccctgagcgattctctggctcccctgactccccttttgggaccacggccaccctgaccatcaccagtgtcgaagccggggatgaggccgactattactgtcatatatgggatagtagagttcccaccaaatgggtcttcggcggagggaccacgctgaccgtgtta

SEQ ID NO: 41 - DNA, 396 bp, Artificial Sequence
  cDNA/PGT122 anti-HIV-1 immunoglobulin heavy chain variable region
caggttcatctgcaggagtcgggccccggactggtgaagccttcggagaccctgtccctcacgtgcaatgtgtctgggaccctcgtgcgtgataactactggagctggatcagacaacccctcgggaagcaacctgagtggattggctatgtccatgacagcggggacacgaattacaacccctccctgaagagtcgagtccacttatcgttggacaagtccaaaaacctggtgtccctgaggctgaccggcgtgaccgccgcggactcggccatatattattgcgcgacaacaaaacacgggaggaggatttatggcgtcgttgccttcaaagagtggttcacctatttctacatggacgtctggggcaaagggacttcggtcaccgtctcctca

SEQ ID NO: 42 - DNA, 315 bp, Artificial Sequence
  cDNA/PGT122 anti-HIV-1 immunoglobulin light chain variable region
acctttgtgtcagtggccccaggacagacggccaggattacttgtggggaagagagccttggaagtagatctgttatttggtatcaacagaggccaggccaggccccttcattaatcatctataataataatgaccggccctcagggattcctgaccgattttctgggtcccctggctccacttttgggaccacggccaccctgaccatcaccagtgtcgaagccggggatgaggccgactattattgtcatatctgggatagtagacgaccaaccaattgggtcttcggcgaagggaccacactgatcgtgtta

SEQ ID NO: 43 - DNA, 396 bp, Artificial Sequence
  cDNA/PGT123 anti-HIV-1 immunoglobulin heavy chain variable region
cagctgcacctgcaggagtcgggcccagggctggtgaagcctccggagaccctgtccctcacgtgtagtgtgtctggcgcctccatcaatgatgcctattggagttggattcggcagtccccagggaagcggcctgagtgggttggatatgtccatcacagcggtgacacaaattataatccctcactcaagaggcgcgtcacgttttcattagacacggccaagaatgaagtgtccctgaaattagtagacctgaccgctgcggactcggccacatatttttgtgcgcgagcacttcacgggaagaggatttatgggatagttgccctcggagagttgttcacctacttctacatggacgtctggggcaaggggactgcggtcaccgtctcctca

SEQ ID NO: 44 - DNA, 315 bp, Artificial Sequence
  cDNA/PGT123 anti-HIV-1 immunoglobulin light chain variable region
tcctctatgtccgtgtccccgggggagacggccaagatctcctgtggaaaagagagcattggtagcagagctgtgcaatggtatcagcagaagccaggccagcccccctcattgattatctataataatcaggaccgccccgcaggggtccctgagcgattctctgcctcccctgacttccgtcctgggaccacggccaccctgaccatcaccaatgtcgacgccgaggatgaggccgactattactgtcatatatatgatgctagaggtggcaccaattgggtcttcgacagagggaccacactgaccgtctta

SEQ ID NO: 45 - DNA, 405 bp, Artificial Sequence
  cDNA/PGT127 anti-HIV-1 immunoglobulin heavy chain region
cagccgcagctgcaggagtcgggcccaggactggtggaggcttcggagaccctgtccctcacgtgcactgtgtccggcgactccactggtcgttgtaattatttctggggctgggtccggcagcccccagggaaggggctggagtggattgggagtttgtcccactgtagaagttactacaatactgactggacctaccacaacccgtctctcaagagtcgactcactatttcactcgacacgcccaagaatcaggtcttcctgagattgacctctgtgaccgccgcggacacggccacttattactgtgcgcgattcggcggcgaagttctagtgtacagagattggccaaagccggcctgggtcgacctctggggccggggaacgctggtcaccgtctcgagc

SEQ ID NO: 46 - DNA, 315 bp, Artificial Sequence
  cDNA/PGT127 anti-HIV-1 immunoglobulin light chain region
cagtctgccctgactcagcctccctccgcgtccgggtctcctggacagtcaatcaccatctcctgcactggaaccagcaataactttgtctcctggtaccaacaatacccaggcaaggcccccaaactcgtcatttatgaggtcaataagcgcccctcaggtgtccctgatcgtttctctggctccaagtctggcagcacggcctccctgaccgtctctggactccaggctgacgatgagggtgtctattattgtagttcacttgtaggcaactgggatgtgattttcggcggagggaccaagttgaccgtccta

SEQ ID NO: 47 - DNA, 405 bp, Artificial Sequence
  cDNA/PGT128 anti-HIV-1 immunoglobulin heavy chain region
cagccgcagctgcaggagtcgggcccaacactggtggaggcttcggagactctgtccctcacctgcgctgtgtccggcgactccactgctgcatgtaattctttctggggctgggtccggcagcccccagggaaggggctggagtgggttgggagtttgtcccattgtgcaagctattggaatcgtgggtggacctaccacaacccgtctctcaagagtcggctcacgcttgctctcgacacacccaagaatctggtcttcctcaaattaaattctgtgactgccgcggacacggccacttactactgtgcgcgattcggcggcgaagttttacgctacacggattggccaaagccggcctgggtcgacctctggggccggggaacgctggtcaccgtctcgagc

SEQ ID NO: 48 - DNA, 315 bp, Artificial Sequence
  cDNA/PGt128 anti-HIV-1 immunoglobulin light chain variable region
cagtctgccctgactcagcctccctccgcgtccgggtctcctggacagtcaatcaccatctcctgcactggaaccagcaataactttgtctcctggtaccagcaacacgcaggcaaggcccccaagctcgtcatttatgacgtcaataagcgcccctcaggtgtccctgatcgtttctctggctccaagtctggcaacacggcctccctgaccgtctctggactccagactgacgatgaggctgtctattactgcggctcacttgtaggcaactgggatgtgattttcggcggagggaccaagttgaccgtccta

SEQ ID NO: 49 - DNA, 423 bp, Artificial Sequence
  cDNA/PGT141 anti HIV-1 immunoglobulin heavy chain variable region
caggtgcagctggtgcagtctgggccggaggtgaagaagcctgggtcctcagtgaaggtctcctgcaaggcctctggaaacaccttcagtaaatatgatgtccactgggtacgacaggccactggacaggggcttgaatgggtgggatggatgagtcatgagggtgataagacagaatctgcacagagatttaagggccgagtcaccttcacgagggacacttccgcaagcacagcctacatggaactgcgcggcctgacatctgacgacacggccatctattattgtacgagaggctcaaaacatcgtttgcgagactacgttctctacgatgactacggcttaattaattatcaagagtggaatgactaccttgaatttttggacgtctggggccatggaaccgcggtcaccgtctcctca

SEQ ID NO: 50 - DNA, 336 bp, Artificial Sequence <220
  cDNA/PGT141 anti-HIV-1 immunoglobulin light chain variable region
gatactgtcgtgactcagtctccactctccctgcccgtcacccctggagaggcggcctccatgtcctgttcgtcgactcagagcctccggcatagtaatggagccaactatttggcttggtatcagcacaaaccggggcagtctccacgactcctaatccgtttaggttctcaacgggcctccggggtccctgacagattcagtggcagtggatcaggcactcattttacactgaaaatcagtagagtggaggctgaagatgctgcaatttattattgcatgcaaggtctgaaccgtccctggacgttcggcaaggggaccaagttggaaatcaaa

SEQ ID NO: 51 - DNA, 423 bp, Artificial Sequence
  cDNA/PGt142 anti-HIV-1 immunoglobulin heavy chain variable region
caggtgcagctggtgcagtctgggcctgaggtgaagaagcctgggtcctcagtgaaggtctcctgcaaggcctctggaaacaccttcagtaaatatgatgtccactgggtacgacaggccactggacaggggcttgaatgggtgggatggattagtcatgagcgtgataagacagaatctgcacagagatttaagggccgagtcaccttcacgagggacacttccgcaaccacagcctacatggaactgcgcggcctgacatctgacgacacggccatttattattgtacgagaggctcaaaacatcgcttgcgagactacgttctctacgatgactacggcttaattaattatcaagagtggaatgactaccttgaatttttggacgtctggggccatggaaccgcggtcaccgtctcctca

SEQ ID NO: 52 - DNA, 336 bp, Artificial Sequence; identical to SEQ ID NO: 50
  cDNA/PGT142 anti-HIV-1 immunoglobulin light chain variable region

SEQ ID NO: 53 - DNA, 423 bp, Artificial Sequence
  cDNA/PGT143 anti-HIV-1 immunoglobulin heavy chain variable region
caggtgcagctggagcagtctggggctgaggtgaagaagcctgggtcctcagtgaaggtctcctgcaaggcctctggaaacaccttcagtaaatatgatgtccactgggtacgacaggccactggacaggggcttgaatgggtgggatggatgagtcatgagggtgataagacagaatctgcacagagatttaaggggcgagtcaccttcacgagggacacttccgcaagcacagcctacatggaactgcgcggcctgacatctgacgacacggccatttattattgtacgagaggttcaaaacatcgcttgcgagactacgttctctacgatgactacggcttaattaattatcaagagtggaatgactaccttgaatttttggacgtctggggccatggaaccgcggtcaccgtctcctca

SEQ ID NO: 54 - DNA, 336 bp, Artificial Sequence
  cDNA/PGT143 anti-HIV-1 immunoglobulin light chain variable region
gatactgtcgtgactcagtctccactctccctgcccgtcacccctggagaggcggcctccatgtcctgtacgtcgactcagagcctccgtcatagtaatggagccaactatttggcttggtaccagcacaaaccagggcagtctccacgactcctaatccgtttaggttctcaacgggcctccggggtccctgacagattcagtggcagtggatcaggcactcattttacactgaaaatcagtcgagtggagcctgaagatgctgcaatttattattgcatgcaaggtctgaaccgtccctggacgttcggcaaggggaccaagttggaaatcaaa

SEQ ID NO: 55 - DNA, 420 bp, Artificial Sequence
  cDNA/PGT145 ant-HIV-1 immunoglobulin heavy chain variable region
caggtgcagttggtgcagtctggggctgaagtgaagaagcctgggtcctcagtgaaggtctcctgcaaggcctctggaaacagtttcagtaatcatgatgtccactgggtacgacaggccactggacaggggcttgaatggatgggatggatgagtcatgagggtgataagacaggcttggcacaaaagtttcagggcagagtcaccatcacgagggacagtggcgcaagtacagtctacatggagttgcgcggcctgacagctgacgacacggccatttattattgtttgaccggctcaaaacatcgcctgcgagattattttctgtacaatgaatatggccccaattatgaagagtggggtgactaccttgcgactttggacgtctggggccatgggaccgcggtcaccgtctcgagc

SEQ ID NO: 56 - DNA, 336 bp, Artificial Sequence
  cDNA/PGT145 anti-HIV-1 immunoglobulin light chain variable region
gaggttgtcataactcagtctccactcttcctgcccgtcacccctggagaggcggcctccttgtcttgcaagtgcagccacagcctccaacattcaactggagccaactatttggcttggtacctgcagagaccagggcaaactccacgcctgttgatccatttggccactcatcgggcctccggggtccctgacagattcagtggcagtggatcaggcacagattttacacttaaaatcagtcgagtggagtctgacgatgttggaacttattattgcatgcagggtctgcacagtccctggacgttcggccaagggaccaaggtggagatcaaa

SEQ ID NO: 57 - DNA, 2000 bp, Artificial Sequence
  HIV (Human Immunodeficiency Virus) ENV GP160
tgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttg